(19) United States
(12) Patent Application Publication
(10) Pub. No.: US 2002/0142349 A1
(43) Pub. Date: Oct. 3, 2002

(54) Laboratory tempering device for tempering at different temperatures

(76) Inventors: Ernst Tennstedt, Hamburg (DE); Lars Peters, Zepernick (DE)

Correspondence Address: SIDLEY AUSTIN BROWN & WOOD LLP, 875 Third Avenue, New York, NY, 10022, US

(21) Appl. No.: 10/015,001

(22) Filed: Feb. 19, 2002

(30) Foreign Application Priority Data
Dec. 12, 2000 (DE) 100 62 889.3

(51) Int. Cl. G01N 33/53; G01N 1/00; C12M 1/34

(52) U.S. Cl. 435/7.1; 435/287.1; 422/50

(57) ABSTRACT

A laboratory tempering device for jointly tempering reaction samples in at least two steps in assigned, specified temperature ranges, which are repeatedly carried out consecutively as sequences of steps, the laboratory tempering device, in a randomly selected first step, at the sequence bringing several first groups of samples, in each case containing at least one sample, to temperatures, which are the same within the group and different between the groups within the first temperature range assigned to the first step, wherein, in a randomly selected step of the sequence, if the reaction product is affected by the two steps with regard to identical evaluation parameters, for at least one of the first groups at least two of the samples belong to different groups, which are brought to temperatures, which are the same within the groups and different between the groups, within the temperature range assigned to the second step, and, if the evaluation parameters are different, at least two random samples belong to different third groups, which are brought to temperatures, which are the same within groups and different between groups, within the second temperature range assigned to the second step.

### FIELD OF INVENTION

The invention relates to a laboratory tempering device of the type named in the introductory portion of claims 1 and 11.

### BACKGROUND INFORMATION AND PRIOR ART

Such devices are used for tempering reaction samples, which are to be brought in a sequence of steps to temperatures in different temperature ranges. The sequence of steps is repeated cyclically in one pass. Such devices are suitable for carrying out special chemical reactions, especially enzyme reactions. The main area of application is the PCR (Polymerase Chain Reaction). For this reaction, the denaturing step is carried out at about 90° C. with, the annealing step at about 50° C. and the elongation step at about 60° C. in the usual three-step method.

In this connection, the determination of the optimum temperature, especially for the annealing step but also for the other steps, is always a problem. For this purpose, experiments at different temperatures are required.

In order to simplify these experiments to find the optimum temperature of a step, the generic laboratory devices, such as those described in U.S. Pat. No. 6,054,263 and in the DE 196 46 115 A1, where developed.

In the case of the state of the art, different temperatures are used in one of the steps, generally the annealing step. The same temperatures are used for the other steps. In the state of the art, the reaction samples are disposed in a two-dimensional array in rows and columns. In the step, in which different temperatures are used, a temperature gradient is applied in one direction of the array, for example, in the direction of the rows. As a result, first groups of samples are formed by the columns, the temperatures being the same within the columns but different between the columns.

When the samples are evaluated after the conclusion of the tempering pass, it is possible to find out the column, in which the results are optimum. The associated temperature is then the optimum temperature of this step, such as the annealing step.

If the temperature gradient is applied to the annealing step and if the temperature range for this step is, for example, 50° to 60° C., temperatures, differing by one degree, can be applied in, for example, 10 columns and, with that, the optimum temperature can be determined.

If the temperatures of the other steps of the sequence are also to be optimized, the same pass must be repeated. Now, however, the gradient is applied to one of the other steps. If all three steps of the usual three-step PCR method are to be optimized, three complete temperature passes must be carried out one after the other. A considerable expenditure of time of about 1.5 hours per pass and a considerable consumption of samples, some of which are extremely expensive, is required for this purpose. Since the optimum temperatures of the individual steps are determined in different passes, possible interactions between the temperatures of the steps are not taken into consideration. As a result, the temperature determined may not be optimum.

In FIG. 5 of the DE 196 46 115 A1, a tempering device, similar to that of claim 11, is described, which applies gradient in different directions (X, Y) to the array of the reaction samples for two steps of the sequence. With this, it is possible to determine the optimum temperature in two temperature ranges in only one pass.

For the last-mentioned device also, a further pass is required to optimize the third temperature. However, in a two-dimensional array of reaction samples, only two directions, X and Y, are available. For a third temperature, a third direction Z would be required, which is not present in a two-dimensional arrangement.

Moreover, according to the state of the act, it was regarded to be indispensable to apply gradients for different steps in different directions, that is, for one step in the direction of the columns and, for the other step, in the direction of the rows, in order to be able to assign the resulting differences unambiguously to the temperature variations in the one or the other step during the investigation of the reaction results.

The main area of application of the generic laboratory tempering devices is in the field of PCR. This usually employs three steps. It would be very advantageous if all three steps could be optimized easily in a single tempering pass. Processes with more than three steps, for which the same problems exist, are also known.

### OBJECT OF THE INVENTION

It is therefore an object of the present invention to provide a laboratory tempering device, which reduces the work and equipment expenditure for the determination of the optimum temperatures of all steps.

### SUMMARY OF THE INVENTION

This objective is accomplished with the distinguishing features of claims 1 and 11.

For the solution of claim 1, the invention starts out from the knowledge that, for most of the processes, which can be implemented on laboratory tempering devices, and especially for the usual three-step PCR process, the temperature changes of the individual steps do not always affect the same evaluation parameter. In the case of the usual PCR process, the temperature changes during the annealing step and during the elongation step essentially affect the same parameter, namely the specificity, that is, the ratio of correctly amplified DNA strands of the correct length to the wrongly amplified strands of a deviating length. The temperature changes during the denaturing step, however, essentially affect a different parameter, namely the yield, that is, the amount of amplified DNA material obtained. These two parameters can be determined independently of one another on the reaction product. If one adheres to a simple example of a tempering device with reaction samples, disposed 2-dimensionally in rows and columns, the laboratory tempering device applies the gradients, which affect the same parameter, in two steps (such as the annealing step and the elongation step) in different directions and the gradients, which affect the different parameters, in two steps (such as the annealing step and the denaturing step) in any direction. In the last-mentioned case, the temperature gradients can even be applied in both steps in the same direction. Since the evaluation parameter of the two steps are different here and can be determined independently of one another, the optimum temperatures for both steps can be determined separately The enormous advantage arises that, in the case of the construction of the DE 196 46 115 A1, which works with the gradients in the X and Y directions, all three steps of the standard PCR process can nevertheless be optimized in one tempering pass, contrary to the previous expectations of those skilled in the art. Since the temperatures of several steps are determined in a common pass, interactions between the steps (cross talk) are also taken into consideration, as is the case, for example, in the annealing step and the elongation step.

The invention is not limited to 2-dimensional arrays of reaction samples in the direction of rows and columns. The reaction samples may also be provided in a three-dimensional arrangement. In that case, three steps can be optimized in different gradient directions. The invention then also offers the advantage that all steps, even in the case of sequences with more than three steps, can also be optimized in one pass, provided that at least one of the steps affects an independent parameter.

To optimize a reaction process, such as the PCR process, not only is the temperature optimization of the individual steps important, but it may also be necessary to optimize the reaction samples with respect to other parameters, such as dilution. The advantage of the invention of being able to carry out temperature optimizations in more steps than are present as independent direction in the arrangement of samples, also offers a solution for this. In a 3-dimensional arrangement, for example, several surfaces can be placed in planes above one another, in which samples of different dilutions are arranged. Since the dilution can affect the same evaluation parameters, it is advantageous if the direction, in which there are different dilutions, is not used for applying different temperatures. The temperature gradients therefore are applied in the planes. It is therefore possible to optimize all three steps of the PCR process with regard to temperatures and also dilution in one pass.

The invention is not limited to the usual arrangement of reaction samples in a thermally conducting tempering block which, for example, is heated and cooled at opposite ends in order to produce a temperature gradient over the block in this manner. Winn individual tempering devices are used for all reaction samples, any arrangements, including also random arrangements of the reaction samples and the individual gradients are possible. In that case, first groups of reaction samples must be tempered appropriately in a first step and second groups of reaction samples must be tempered in accordance with the directions of claim 1 in the second step. In the case of a random arrangement of reaction samples and/or of the temperature steps, these groups may be distributed randomly over the sample arrangement. With present-day computer technology, the resulting complicated linkage of the samples to the first and second groups does not represent a problem.

If the evaluation parameters are at least largely independent of one another, which is the case, as mentioned, with the usual PCR process, for example, for the annealing step and the denaturing step, the samples of the third group can, as already mentioned, be selected completely at random, since the group composition of the other steps does not have to be taken into consideration because of the independent evaluation of the parameters.

The distinguishing features of claim 2 are then provided advantageously. The pattern of group formation for the first groups can be used here for forming the third groups. This simplifies the tempering device appreciably. In the case of the already mentioned standard application case of a two-dimensional array in rows and columns, the first groups may consist, for example, of rows and the third groups once again also of rows. The control geometry of the first step can therefore be taken over for the second step. If one temperature gradient is used in a thermally conducting block, the direction of the gradient of the second step can be parallel or anti-parallel to the direction of the gradient of the first step. Among other things, this means that, with a simple laboratory tempering device, which can produce a gradient in only one direction, both steps of a two-step method can nevertheless be optimized if the evaluation parameters of these two steps are different.

Pursuant to the invention, it is generally only necessary that, for only one of the first groups, at least two of the samples belong to different second groups. With that, very simple, roughly screened optimizations can be carried out, which investigate, for example, only two different temperatures per step. Advantageously, however, the distinguishing features of claim 3 are provided, according to which, for example, even in the case of larger groups, all samples of the second groups belong to different first groups. In the case of a two-dimensional array with the rows and columns, this would mean that, for example, the temperatures between the columns are different in the first step and the temperatures between the rows are different in the second step, so that all samples of a column lie in different rows (second groups).

As already mentioned, the invention can also be carried out with a very complex arrangement of the reaction samples. Advantageously, however, the distinguishing features of claim 4 are provided. This permits the inventive laboratory tempering device to be constructed in the usual standard manner, as known, for example, from one of the publications named above. Advantageously, the distinguishing features of claim 5 are also provided, so that the conventional, easily surveyed orthogonal arrangement is used, which has the advantage that the gradients can be produced in the direction of rows and columns, the orthogonal edges of a block. According to claim 6, the groups are then assigned advantageously to the rows and columns.

Generic laboratory tempering devices are used not only to optimize the temperatures of the individual steps, but also to provide for the mass processing of samples after the optimum temperatures have been determined. They therefore accommodate a very large number of reaction samples, such as 384 samples in 24 columns and 16 rows.

Moreover, the distinguishing features of claim 7 are provided advantageously. According to these, if, for example, only one of the first groups contains several samples and also only one of the second groups contains several samples and the samples are in a two-dimensional array with rows and columns, only one of the rows and one of the columns, for example, is occupied completely by samples, that is, there is only occupied row per direction. If gradients are applied in the direction of rows and columns for the steps, the temperature can be optimized with very few samples, that is, with a very economical consumption of the expensive samples. Moreover, the laboratory tempering device can certainly be constructed to accommodate a very large number of samples, such as 384 samples. The rest of the sample spaces remain unoccupied. However, it is also possible to use a special device, which has only one sample column and one sample row and which is provided especially only for optimization and not for a mass throughput. Of course, it is also possible to use more than one occupied row per direction, for example, two parallel rows next to one another or also at a distance from one another.

Furthermore, the distinguishing feature of claim 8 is advantageously provided. This distinguishing feature ensures that, for steps in which its samples are at the same temperature, each group is located in the central region of the assigned temperature range. For example, in the annealing step, the optimum temperature is sought with different temperatures. For the other steps, however, the average temperatures of the temperature range are used in order to ensure informative results.

If different evaluation parameters are affected in two strips and the evaluated results, therefore, are independent, the third groups can also be selected at random. In accordance with claim 2, they may also coincide with the first groups. Advantageously,they can also be selected in accordance with claim 9, according to which the third groups, in the case of a two-dimensional array of reaction samples, are divided into coherent areas, which are occupied only with, in each case, samples of one group. This makes a particularly simple evaluation possible. If the samples are disposed in rows and columns in the tempering device, the third groups, deviating from the rows and columns, can be disposed in areas, such as four sectors, provided that the construction of the tempering device permits this. If the reaction samples are tempered individually, the areas assigned to the third groups, can be selected at random. For example, for thermal reasons, warmer groups can be placed in the interior of the array and colder groups at the edge.

In the case of multi-step sequences of steps, the invention can be applied to only a few of the steps, such as two of three, the third step being carried out without temperature optimization with all the reaction samples at the same temperature. However, the distinguishing features of claim 10 are provided advantageously. As already mentioned, the temperature optimization represents an appreciable advantage for all steps of a process.

For the invention of claim 11, the reaction samples are assigned to first, second and third groups, which in each case. For one of the steps in the associated temperature range, have different temperatures between the groups but the same temperatures within the groups. With this, it is possible to work in each step with different temperatures in different group divisions. Accordingly, the optimum temperatures can be determined for all three steps of the conventional PCR process in one pass.

The samples can also be disposed three-dimensionally, three dimensions being formed. In the case of an orderly arrangement, columns, rows and planes, for example, are formed, in which the samples can be grouped in a clear manner. As already mentioned above in connection with claim 1, irregular, 3-dimensional arrangements with correspondingly complicated group sub-divisions are also possible, if individual heating is provided for the reaction samples.

Advantageously, the distinguishing features of claim 12 are provided. With this very simple arrangement with temperature-gradients in the X, Y and Z directions, it is possible to work in a conventional, thermally conducting block, in which the reaction samples are disposed. However, compared to the conventional arrangement, the tempering block must be constructed in three dimensions and so that it can be heated.

The distinguishing features of claim 13, according to which the reaction samples are disposed in a surface, are also advantageous. For example, several partial surfaces, which have the usual X-Y arrangement of samples and which correspond to the planes of a three-dimensional arrangement, can be disposed, for example, next to one another in a surface. In the computer, which controls the laboratory tempering device, the two-dimensional arrangement, which is somewhat unclear, can be recalculated for representation purposes into the well-ordered three-dimensional arrangement with three coordinates.

The distinguishing features of claim 14 are provided advantageously. In each case, gradients are applied in opposite directions in partial surfaces in two steps, the partial surfaces of the two steps overlapping in quadrants. In the third step, the quadrants formed are brought to different temperatures. In this way, it is also very easily possible to optimize the temperatures in three steps for all steps in one pass in a two-dimensional arrangement of reaction samples.

The distinguishing features of claims 1 to 10 as well as of claims 11 to 14 can also be combined in an advantageous manner.

The invention is shown by way of example and diagrammatically in the drawings, in which

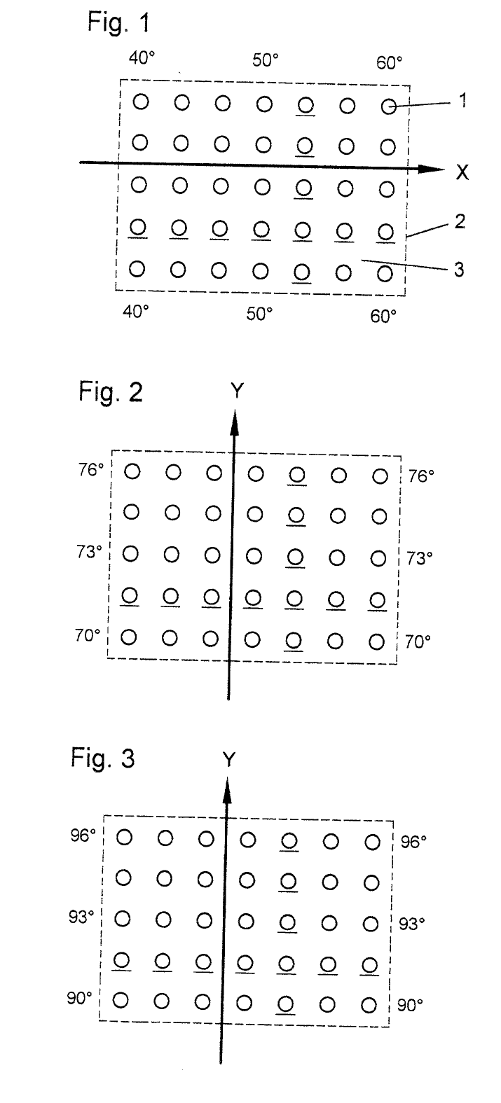

FIG. 1 shows a highly diagrammatic, inventive laboratory tempering device in plan view of two-dimensional array of reaction samples, disposed in rows and columns, with a temperature gradient for the annealing step of a standard PCR process applied in the X direction,

FIG. 2 shows the view of FIG. 1 for the elongation step with a temperature gradient in the Y direction,

FIG. 3 shows a view of FIG. 1 for the denaturing step with a temperature gradient also in the Y direction,

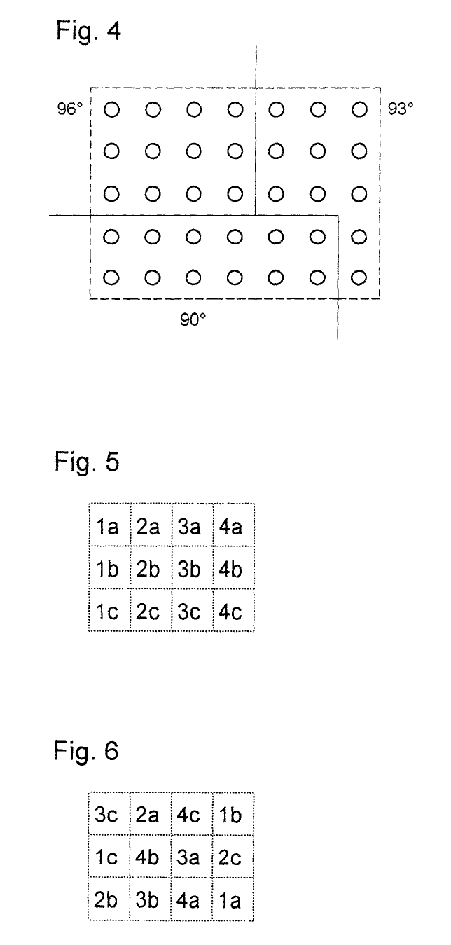

FIG. 4 shows a view of FIG. 1 for the denaturing step with the array divided into three two-dimensional groups,

FIG. 5 shows a diagrammatic representation of an array of reaction samples, which are ordered in rows and columns and assigned to the first groups (numbers) and second groups (letters),

FIG. 6 shows a representation of FIG. 5 with a different arrangement of the reaction samples,

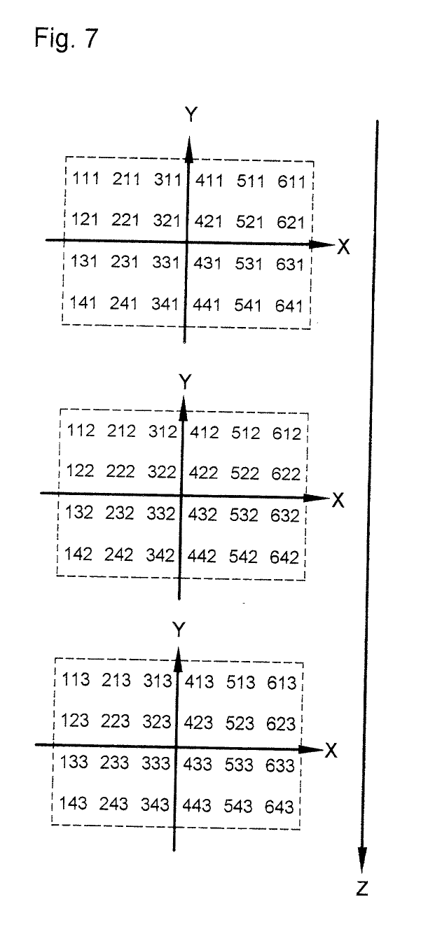

FIG. 7 shows a highly diagrammatic, inventive laboratory tempering device with a three-dimensional arrangement of reaction samples, the planes of the three-dimensional arrangement being shown on top of one another in the Figure,

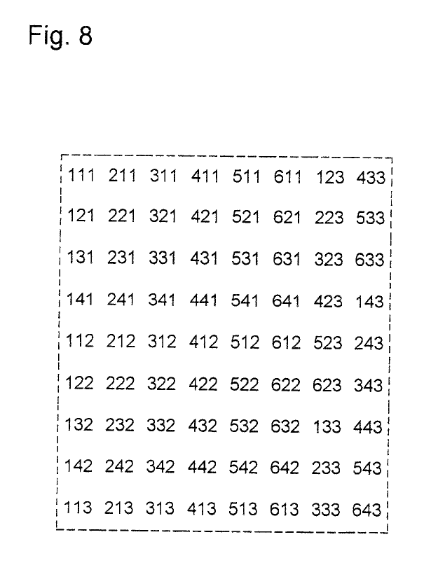

FIG. 8 shows the representation of an arrangement of the samples of the embodiment of FIG. 7 in a two-dimensional array and

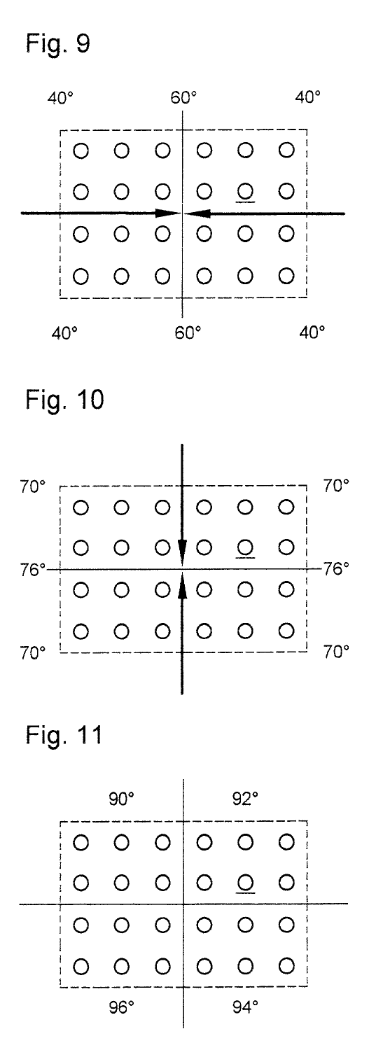

FIGS. 9 to 11 show a further embodiment of the laboratory tempering device with the temperatures set in three steps.

FIG. 1 shows a plan view of an array of a total of 35 reaction samples 1, which are disposed in a two-dimensional array in orthogonal rows and columns. The field is bounded by a border 2, which is shown by broken lines. It may, for example, be a conventional tempering block 3, which is bounded by the border 2, as explained, for example, in FIG. 5 of the DE 196 46 115 A1, with the possibility of applying a temperature gradient in the direction of the rows or in the direction of the columns. For the technical details in this connection, reference is made to the publication cited.

In FIG. 1, the laboratory tempering device is operated to carry out the annealing step of the standard PCR process. The temperature gradient, shown by the arrow pointing in the row direction, is applied in the X direction. It ensures that all reaction samples 1 of the first column are at 40° C., all samples of the last column are at 60° C. and those of the middle column are at 50° C. The remaining columns have temperatures in between.

The reaction samples 1, which are shown, accordingly are tempered differently in first groups, the first groups corresponding to the columns of the arrangement shown. Within each first group (column), all reaction samples 1 have the same temperature. Between the columns, there are different temperatures.

FIG. 2 shows the tempering device of FIG. 1 during the elongation step. A temperature gradient in the sense of the arrow shown is applied in the Y direction here with suitable devices, which are not shown. The lowest row is at 70° C. and the uppermost at 76° C. The intermediate rows have corresponding, intermediate temperatures. In other words, the reaction samples in second groups are kept here at different temperatures, the second groups corresponding to the rows. From a comparison of FIGS. 1 and 2, it can be seen that, for the two steps shown in FIGS. 1 and 2, in each case all samples of a first group (column) belong to different groups (rows) and vice-versa. In the two-dimensional representation, this means that the groups and also the temperature gradients are orthogonal to one another.

FIG. 3 shows the same tempering device while carrying out a third step, namely the denaturing step. This step also is to be optimized to the most advantageous temperature and, moreover, in a temperature range which, in the examples shown, is between 90° C. and 96° C. The temperature gradient is applied here in the Y direction.

The three steps of annealing, elongation and denaturing form a sequence of steps, which is repeated several times for an exponential amplification.

As shown in FIGS. 1 to 3, the temperature is varied in all steps in the temperature range assigned to the step. According to FIG. 1, different first groups (columns) are kept at different temperatures between 40° C. and 60° C. According to FIG. 2, second groups (rows) are kept at different temperatures ranging from 70° C. to 76° C. According to FIG. 3, different third groups (once again rows) are kept during the denaturing step at temperatures between 90° C. and 96° C. Different temperatures are applied in all three steps. By evaluating the result of the reactions at the end of the pass, it is possible to find out which temperature is optimum in which step. The result of reaction can also be followed continuously during the pass (online monitoring).

This evaluation does not represent a problem for the two steps of FIGS. 1 and 2, since the gradients are in the X and Y directions and are therefore orthogonal to one another. It is merely necessary to determine the reaction sample with the best result, after which one can see from the row and the column what is the best annealing temperature (FIG. 1) and what is the best elongation temperature (FIG. 2). For the denaturing step of FIG. 3, the third groups, which were tempered differently there, coincide with the second groups, which were tempered differently in FIG. 2. Both these groups are rows.

Different annealing temperatures affect essentially the specificity of the reaction result. Specificity is defined as the ratio of the correctly amplified DNA pieces of the correct length to the incorrectly amplified DNA pieces of deviating length. The elongation temperature affects essentially the same evaluation parameter, namely the specificity. However, the denaturing temperature in the step of FIG. 3 affects essentially the yield, that is, the amount of reaction material obtained.

The tempering device therefore is constructed so that, for the two steps of annealing (FIG. 1) and elongation (FIG. 2), which affect the same evaluation parameter, the temperature gradients are applied in independent directions X and Y. For the step of FIG. 3 (denaturing), which affects a deviating evaluation parameter, namely the yield, the temperature gradient can be applied in any direction. It is in the Y direction for the example shown in FIG. 3. It can, however, also be in the X direction.

In a variation of the embodiment, FIG. 4 shows the device of FIGS. 1 to 3 during the denaturing step, that is, at temperatures ranging from 90° C. to 96° C. However, the third groups of different temperature are disposed not in rows or columns, but in the form of the three area regions shown, which are at three temperatures, 90° C., 93° C. and 96° C. The areas are divided by the range boundaries shown.

The embodiment of FIG. 4 presupposes a somewhat different construction. A block having good thermal conductivity, which is suitable for applying temperature gradients in the X and Y directions, as used for the embodiment of FIGS. 1 to 3, would not be very suitable for forming the well defined areas of FIG. 4, which are tempered uniformly. However, special constructions can do this, especially devices with individual tempering of the individual reaction samples 1. Such a construction of the laboratory tempering device can then of course also produce the temperature gradients, which are shown in FIG. 1 to 3.

The invention is not limited to the embodiment shown in FIGS. 1 to 4.

FIG. 5 once again, highly diagrammatically, shows a two-dimensional array of reaction samples, which are disposed in rows and columns. Each reaction sample is shown with a number/letter combination. The numbers refer to the columns and the letters to the rows. A reaction sample in the second row and the third column therefore is labeled 3b.

FIG. 6 shows the same reaction samples, which are shown in FIG. 5, however in a different, for example, random arrangement. With such an arrangement also, which presupposes, however, individual tempering of the reaction samples, a laboratory tempering device can also be operated pursuant to the invention. With the help of a computer, for example, it must ascertain first groups (such as the numbers 1 to 4) and temper them differently during a first step but the same internally and, in a second step, it must temper second groups (letters) differently groupwise but with the same temperatures within groups. If the evaluation parameters are different in the two steps, the device can form any groups in the two steps and temper them appropriately.

In the embodiments shown, the reaction samples are sorted in rows and columns in a two-dimensional array. This makes it easier to use especially conventional tempering blocks, which are suitable only for forming temperature gradients in orthogonal directions, that is, in the direction of the columns or the rows. For a different construction of the device, especially if the device is equipped with individual tempering for the individual reaction samples, completely random arrangements, deviating from the row and column pattern, can also be selected.

The invention is not limited to devices with a two-dimensional arrangement of the reaction samples. The reaction samples can also be arranged three-dimensionally, for example, in a three-dimensional lattice. In that case, three steps, which all affect the same evaluation parameter, can be optimized simultaneously with respect to their temperature. In the case of a tempering process, which has more than three steps, directions, which have already been used, can be used once again for the additional steps, provided that the evaluation parameters are independent. With individual heating of the samples, the three-dimensional arrangement mentioned can also be re-sorted, as explained by means of the two-dimensional example in FIGS. 5 and 6. An arrangement in one plane, on which the three-dimensional arrangement is depicted, is also possible.

In the example of FIGS. 1 to 3, an array of samples 1 is shown, which forms 5 rows and 7 columns and therefore has a total of 35 samples. Accordingly, in order to determine the optimum temperatures for the three steps in one pass, 35 expensive samples must be used.

Economizing is possible, in that, as shown in FIGS. 1 to 3, only one column and one row is occupied by samples. This is shown in FIGS. 1 to 3 by underlining the samples in the fifth column and fourth row.

The resulting cross arrangement enables the effect of the temperature gradient applied in the X as well as in the Y direction to be determined in each case by one row of samples. The unoccupied sample places can remain empty. In a special version, the laboratory tempering device can also be constructed only for the purpose of optimizing the temperature and in that case have only the sample spaces shown underlined in FIGS. 1 to 3.

FIG. 7 shows a further embodiment of a laboratory tempering device, for which the reaction samples are disposed in an orthogonal, three-dimensional arrangement with six columns, four rows and three planes. The three planes, which actually are one above the other, are shown next to one another in FIG. 7, in order to simplify the overall view. Reaction samples, which are shown by three digit numbers in FIG. 7, are provided at the places of the arrangement shown. In each case, the first digit refers to the column, the second digit to the row and the third digit to the plane. Therefore, in the plane at the bottom of FIG. 7, all numbers end with a 3 because this is the third plane.

In the example, the samples are disposed in a three-dimensional block of a thermally conductive material. Gradients can be applied in the X, Y or Z direction to this block in a manner known from the art. If a gradient is applied in the X direction, samples with a lower column number are at a lower temperature and samples with a higher column number are at a higher temperature. If the gradient is applied in the Y direction, it extends transversely to the rows and brings these to different temperatures. If the gradient is in the Z direction, the planes are brought to different temperatures. At the same time, the rows, columns or planes, which extend obliquely to the gradient applied, are in each case at the same temperature.

In the case of a process with a three-step sequence, with the laboratory tempering device shown with a cyclically repeated sequence of steps, it is possible, for example, to apply one gradient time and again in the X direction in the first step, another in the Y direction in each second step and a third in the Z direction in each third step.

The three-dimensional arrangement shown in FIG. 7, with an orthogonally disposed arrangement of reaction samples, is distinguished by great clarity. If the samples are tempered with individual heating and not in a thermally conductive block, deviating three-dimensional arrangements can also be used, for which the samples are exchanged, for example, randomly, similarly to the exchange between FIGS. 5 and 6.

FIG. 8 shows a variation of the embodiment, for which all the samples, shown in FIG. 7, are disposed in the two-dimensional array shown. It can be seen that the three planes, shown individually in FIG. 7, are disposed next to one another in one plane here and, moreover, in the first six columns above one another, the upper and middle area according to FIG. 7 and, in the s eve nth and eighth column, the lowest surface of FIG. 7 in the re-arrangement. With individual heating of the reaction samples or, for example, suitable subdivision of larger heating devices, which are not shown, the same gradients which were explained in connection with the embodiment of FIG. 7 can be applied stepwise to the samples of the arrangement of FIG. 8.

FIGS. 9 to 11 show a further embodiment of the laboratory tempering device in three steps of a three-step sequence. FIG. 9 shows the annealing step, FIG. 10 the elongation step and FIG. 11 the denaturing step. The temperatures, given in the Figures, correspond to the associated temperature ranges, which were already explained by means of FIGS. 1 to 4.

In all three FIG. 9 to 11, the same two-dimensional array of reaction samples, which are in each case indicated by circles, is shown. In the example, the reaction samples are disposed in six columns and four rows in an orthogonal alignment.

For the annealing step of FIG. 9, the array surface is divided with a first perpendicular center line into two partial areas (to the left and right of the center line). The temperature gradients, which are represented with arrows and lead to the temperature distribution given by the numbers, are applied in the two partial surfaces so formed. The outermost columns on the right and left are at 40° C. and those close to the first center line are at 60° C. In other words, the gradients are the same, but extend in opposite directions.

During the elongation step of FIG. 10, a corresponding temperature distribution is applied. However, this temperature distribution falls within the range of 70° C. to 76° C., which is required for the elongation step. However, the second center line is perpendicular here to the first center line, that is, it is horizontal. Opposite but equal gradients are applied once again in the second partial surfaces so formed.

FIG. 11 shows the denaturing step. Different temperatures, from 90° C. to 96° C., that is, temperatures in the range required for denaturing, are applied in the four quadrants, which are formed from the two middle lines, which can be seen in FIGS. 9 and 10.

If the reaction sample, which is underlined and lies in column 5, row 2, is considered and followed through the three steeps, shown in FIGS. 9, 10 and 11, it can be seen that this sample can be identified unambiguously with respect to the optimum temperatures in the three steps. For the annealing step of FIG. 9, it requires a temperature of about 50° C., for the elongation step of FIG. 10, a temperature of about 70° C. and for the denaturing step of FIG. 11, a temperature of about 92° C.

If the optimum temperature for the step of FIG. 9 were at 50° C., for the step of FIG. 10 at 75° C. and for the step of FIG. 11, however, at a different temperature, such as 96° C., then the sample, for which all three temperatures are optimum, would be located in the second column and the third row. It should be noted that, if the optimum temperature in FIG. 9, as mentioned, is 50° C., then this optimum temperature exists in the fifth row as well as in the second row. For the sample selected of FIG. 10, the optimum temperature is in the second row and also in the third row.

It should be noted that only a very small array surface with few reaction samples is shown in FIGS. 9 to 11, in order to make the diagrammatic representation easier. As a result, there is only one differentiation for the reaction samples into temperatures in the elongation step of FIG. 10. If the number of rows and columns is increased clearly, significantly finer temperature differences can be evaluated.

If the usual, significantly larger number of 384 samples is used in 24 columns and 16 rows instead of the array surface with 24 samples in 6 columns and 4 rows, shown in FIGS. 9 to 11 and partial surfaces in the form of quadrants are employed, as shown in FIGS. 9 to 11, four quadrants with 12 columns and 8 rows each result. If temperature transition problems are to be avoided in the region of the middle lines between the quadrants, the two rows or columns, adjacent to the center lines in each quadrant, may, for example, be left unoccupied. Regions, which are occupied with samples and in each case take up 60 samples in 10 columns and 6 rows and in which the optimum temperature can be sought with a high temperature resolution, then remain disposed in the four corners of the array.

If such a laboratory tempering device is used, with a continuously thermally conducting tempering block of the construction shown in FIGS. 1 to 3 of the DE 196 46 115 C2, the tempering block can be occupied, for example, by nine Peltier elements in a 3×3 arrangement over a large area at its underside, which is averted from the samples. In both directions, the middle Peltier elements lie below the middle line and, in each case, heat two adjacent quadrants from the edge. With this arrangement, the gradient tempering of FIGS. 9 and 10 can be achieved alternately by acting on the Peltier elements with different electrical currents. To produce the heating of FIG. 11, which differs from quadrant to quadrant, the underside of the tempering block could be provided additionally with heating foil, which covers the quadrants and bring the quadrants individually to the desired temperature during the step of FIG. 11 and with the Peltier elements switched off

Contrary to the embodiment shows in FIGS. 9 to 11, the gradients can also be applied in a different way. According to the representation in FIG. 9, the top right and bottom right quadrants are acted upon with the same gradient in the same direction. In these two quadrants, it would also be possible to apply the gradient in opposite directions. The same holds good for the top left and the bottom left quadrants. For example, the gradient can be applied in the top left quadrant with the arrow to the left and in the bottom left quadrant with the arrow to the right. The same applies also for FIG. 10.

It is generally the case for this embodiment that gradients are applied in different directions in the first and second steps in the partial surfaces formed by the quadrants and that all samples of a partial surface are at the same temperature in the third step (FIG. 11). If, for example, six different temperatures are required in the third step and not the four, which are shown in FIG. 11, six partial surfaces, which are to be treated in the specified manner, are accordingly required.

In the third step, there is a different temperature in each partial surface. In the other two steps, gradients are applied over each partial surface. Since there is a sample for each combination of the different temperatures of the different steps and this sample was treated with this temperature combination, all interactions between the steps are also taken into consideration with this arrangement.

1. A laboratory tempering device for jointly tempering reaction samples in at least two steps in assigned, specified temperature ranges, which are repeatedly carried out consecutively as sequences of steps, the laboratory tempering device, in a randomly selected first step of the sequence bringing several first groups of samples, in each case containing at least one sample, to temperatures, which are the same within the group and different between the groups within the first temperature range assigned to the first step, wherein, in a randomly selected step of the sequence, if the reaction product is affected by the two steps with regard to identical evaluation parameters, for at least one of the first groups at least two of the samples belong to different groups, which are brought to temperatures, which are the same within the groups and different between the groups, within the temperature range assigned to the second step, and, if the evaluation parameters are different, at least two random samples belong to different third groups, which are brought to temperatures, which are the same within groups and different between groups, within the second temperature range assigned to the second step.

2. Laboratory tempering devices of claim 1, wherein, in the case of two steps with different evaluation parameters, all samples of a third group are contained in a first group or all samples of a first group are contained in a third group.

3. The laboratory tempering devices of claim 1, wherein, in the case of two steps with identical evaluation parameters, all samples of all second groups belong to different first groups.

4. The laboratory tempering devices of claim 1, wherein, in the case of all steps of the sequence, the samples are brought groupwise to different temperatures in the assigned temperature range.

5. The laboratory tempering devices of claim 1, wherein only one of the first and/or second and/or third groups contains several samples.

6. The laboratory tempering devices of claim 5, wherein groups, which contain several samples, contain those of other groups which, in their assigned temperature range, are in the vicinity of the middle temperature.

7. The laboratory tempering devices of claim 1, wherein the samples are disposed in an array of rows and columns.

8. The laboratory tempering devices of claim 7, wherein the rows and columns are disposed orthogonally to one another.

9. The laboratory tempering devices of claim 7, wherein all samples of a group are in a row or in a column.

10. The laboratory tempering devices of claim 7, wherein the third groups form partial regions of the array, which enclose, with their regional boundaries, only samples of the same third group.

11. A laboratory tempering device for jointly tempering reaction samples in at least three steps in assigned, specified temperature ranges, which are repeatedly carried out consecutively as sequences of steps, the laboratory tempering device, in a randomly selected first step of the sequence bringing several first groups of samples, in each case containing at least one sample, temperatures, which are the same within the group and to different temperatures between the groups within the first temperature range assigned to the first step, and, in a randomly selected second step of the sequence, in the case of at least one of the first groups, at least two samples in each case belong to different second groups, which are brought to temperatures, which are the same within groups and different between groups, of the second temperature range assigned to the second step, wherein in a randomly selected third step of the sequence, in the case of at least one of the first groups and at least one of the second groups, at least two samples belong to different third groups, which are brought to temperatures, which are the same within the groups and different between the groups, within the third temperature range assigned to the third step.

12. The laboratory tempering device of claim 11, wherein the reaction samples are disposed three-dimensionally and temperature gradients are applied in the X, Y and Z directions for producing the different temperatures for three steps.

13. The laboratory tempering device of claim 11, wherein the reaction samples are disposed in a surface.

14. The laboratory tempering device of claim 13, wherein, in the first step, the surface is divided with a first center line into two first partial areas, in which, in each case, identical temperature gradients are applied in opposite directions perpendicularly to the center line, and wherein, in the second step, the surface is divided with a second center line, which is perpendicular to the first center line, into two second partial areas, in which, in each case, identical temperature gradients are applied in opposite directions perpendicularly to the center line, and wherein, in the first step, different temperatures are applied in the four quadrants of the surface, which are formed by the two center lines.
